(19) United States
(12) Patent Application Publication
(10) Pub. No.: US 2011/0268716 A1
(43) Pub. Date: Nov. 3, 2011

(54) METHODS FOR THE ENRICHMENT OF VIABLE FOXP3+ CELLS AND USES THEREOF

(75) Inventor: Song-Guo Zheng, Arcadia, CA (US)

(73) Assignee: UNIVERSITY OF SOUTHERN CALIFORNIA, Los Angeles, CA (US)

(21) Appl. No.: 13/098,232

(22) Filed: Apr. 29, 2011

Related U.S. Application Data
(60) Provisional application No. 61/329,784, filed on Apr. 30, 2010.

(51) Int. Cl. A61K 35/12; C12Q 1/02; A61P 37/02; C12N 5/0783

(52) U.S. Cl. 424/93.71; 435/325; 435/29

(57) ABSTRACT

The present invention is directed to methods of identifying and enriching for viable Foxp3⁺ cells and the use of such cells. In particular, the present invention provides methods whereby viable Foxp3⁺ cells are isolated from a mixed population of cells; Foxp3⁺ cells being identifiable as those cells with relatively high forward scatter as assessed by a flow cytometer.

### CROSS REFERENCE TO RELATED APPLICATIONS

This application claims the benefit of priority to 61/329,784, filed Apr. 30, 2010, which is hereby incorporated by reference in its entirety for all purposes.

### STATEMENT REGARDING FEDERALLY SPONSORED RESEARCH

This invention was made with U.S. Government support under Grant No. R01AR059103 awarded by the National Institutes of Health. The U.S. Government has certain rights in this invention.

### BACKGROUND OF THE INVENTION

CD4⁺ T cells comprise a heterogeneous population of T cells which are of fundamental importance in both the generation of immune responses and the suppression of autoimmune diseases. A subpopulation of CD4⁺ T cells expresses the transcription factor forkhead box P3 (Foxp3). This subpopulation, loosely defined as regulatory T cells or Treg(s), plays a pivotal role in maintaining self tolerance.

Tregs are functionally defined as T cells that inhibit the immune response by influencing the activity of another cell. These cells comprise a small population of thymus derived CD4⁺ T cells. Despite there being a small population, Tregs have a large regulatory effect on the immune system.

Tregs mediate inflammatory and autoimmune disorders. For example, Tregs play a role in preventing autoimmune gastritis, thyroiditis, insulin-dependent diabetes melitus (IDDM), inflammatory bowel disorders (IBD), experimental autoimmune encephalomyelitis (EAE), food allergies, and graft rejection.

Conversely, impaired Treg activity can promote autoimmune disorders, see, e.g., Wing, et al. (2003) Eur. J. Immunol. 33:579-587; Sakaguchi, et al. (2001) Immunol. Revs. 182:18-32; Sufi-Payer, et al. (1998) J. Immunol. 160:1212-1218; Shevach (2001) J. Exp. Med. 193:F41-F45; Read and Powrie (2001) Curr. Op. Immunol. 13:644-649.

Because of these properties, there has been increasing interest in the possibility of using Tregs in immunotherapy to treat or prevent autoimmune diseases, allergies and transplantation-related complications, such as graft rejection or graft-versus-host disease (GvHD) (For a review, see Roncarolo, M. G. & Battaglia, M., Nat Rev Immunol 7, 585-98 (2007)).

The characteristic marker for Treg cells is Foxp3. Foxp3 is a transcription factor that is expressed in the nucleus; thus, it is not possible to directly isolate viable Foxp3⁺ cells using anti-Foxp3 antibodies.

Because of this, a number of methods using cell surface markers associated with, but not restricted to, Tregs have been employed for their isolation. see, e.g. Seddiki, N. et al., (2006) J Exp Med 203, 1693-700. These markers include CD4⁺, CD25 (IL-2 receptor alpha chain), CTLA-4 (cytotoxic lymphocyte-associated antigen 4), GITR (glucococorticoid-induced lymphocyte-associated antigen-4), and lymphocyte activation gene-3.

Downregulation of CD127, the α chain of the IL-7 receptor, has been described as useful in the discrimination of Tregs from conventional T helper cells (Tconv) (Seddiki et al., 2006), but unfortunately CD127 expression is also down-regulated upon T cell activation and the loss of CD127 may be a characteristic feature of both activated Tconv cells, and follicular helper T cells which provide help for B cells (Lim and Kim, 2007).

For instance, Hoffmann, P. et al. (2006) Biol Blood Marrow Transplant 12, 267-74 describe the isolation of CD4⁺CD25⁺ T cells with regulatory function from standard leukapheresis products by magnetic cell-separation. Approximately half of the cells isolated this way are Foxp3⁺ Treg cells.

Contamination of the isolated Treg subsets with effector T cells possesses a significant risk. Effector T cells drive pro-inflammatory immune reactions by secreting cytokines such as IFN-γ or IL-17. A fatal immune response was recently documented in the failure of the “Tegenero” trials, where an antibody expected to expand Treg cells led to an activation of effector cells (Suntharalingam, G. et al., N Engl J Med 355, 1018-28 (2006)).

From the foregoing it follows that there is a particular need for methods useful for isolating Foxp3⁺ Treg cells which are substantially free from contaminating cells.

It is therefore an object of the present invention to provide a method which avoids the above mentioned disadvantages of the prior art. In particular, it is an object of the present invention to provide a method for the isolation of viable immune-suppressive Foxp3⁺ Treg cells while allowing for the effective removal of contaminating cells.

These and other advantages will be described below.

### SUMMARY OF THE INVENTION

Accordingly, the present invention provides methods of enrichment for Foxp3 expressing cells, compositions of such enriched cells, and methods of their use.

In some embodiments, a method for enriching for Foxp3 expressing cells is provided, the method encompassing providing a population of lymphocytes, isolating from among the population of lymphocytes those cells with relatively high forward scatter and relatively low side scatter, and thereby enriching for Foxp3 expressing cells. The population of lymphocytes is preferably (1) an expanded population of nTregs, or (2) a population of iTregs made from naïve T cells with IL-2 and TGF-β.

In other embodiments, a composition of cells enriched for Foxp3 expression is provided, wherein more than 75% of cells of the composition express Foxp3.

In another embodiment, a method of treating a subject is provided, the method encompasses providing a population of lymphocytes as above, isolating from among the population of lymphocytes cells that express Foxp3, preferably those cells with relatively high forward scatter and relatively low side scatter, and administering the isolated cells to a subject in need.

### BRIEF DESCRIPTION OF THE DRAWINGS

FIG. 1 shows FoxP3 expression of fresh or expanded nTregs. (A) Foxp3 expression on fresh nTregs in spleen on CD25^{bright} gate; (B) spleen CD4⁺CD25⁺ cells in Foxp3^{gfp} knock-in mice (C57BL/6 strain) were stimulated with anti-CD3/CD28 coated beads (one bead to five cells) in the presence of IL-2 (200 units/ml) for four days. Foxp3 expression on expanded nTregs on CD25^{bright} and CD127^{-/dim} gates. All data shown in Figure A and B represent at least four separate experiments.

FIG. 2 shows lymphoblast population of expanded CD4⁺CD25⁺ nTregs in scatter plot identifies purified and viable Foxp3⁺ cells. (A) CD4⁺CD25⁺ cells in Foxp3^{gfp} knock-in mice (C57BL/6 strain) were stimulated as FIG. 1B. Foxp3 (GFP) expression on total expanded nTregs, non-lymphoblast or lymphoblast cell populations was determined by flow cytometry. (B) T cells were isolated from C57BL/6 mice and stimulated with anti-CD3 (0.25 μg/ml) in the presence of γ-irradiated (20 cGy) antigen-presenting cells (1:1 ratio) for three days. Total expanded nTregs, non-lymphoblast or lymphoblast cell populations sorted as FIG. 2A were added to some culture wells (one conditioned cell to four T responder cells). ³H (1 μCi) was added to each well (96-well plates) in the final 16 hours of cultures and the proliferation was determined by ³H incorporation using a liquid scintillation counter. All data shown in Figure A and B represent at least four separate experiments.

FIG. 3 shows Lymphoblast population of activated iTregs in scatter plot also identifies purified and viable Foxp3+ cells. (A) Scatter plot of iTreg induction in the different time points. Naïve CD4⁺GFP⁻ cells isolated from Foxp3^{gfp} knock-in mice were stimulated with anti-CD3/CD28 beads (1:5) and IL-2 (40 U/ml) and TGF-β (2 ng/ml) for 1-4 days. (B) Treg cell relative phenotypes were analyzed and compared between total cell and two distinct cell populations. Data is representative of three independent experiments.

FIG. 4 shows Purified Foxp3+ cells obtained from lymphoblast cell population display more potent suppressive activity in vitro and in vivo. (A) Lymphoblast, non-lymphoblast and total cell population were stimulated with soluble anti-CD3 (0.25 μg/ml) in the presence of irradiated APC (1:1) with or without IL-2 (50 μg/ml) for three days. The proliferation was determined by ³H incorporation. (B) These cell were added T responder cells (1:4 ratio as in FIG. 2B) and stimulated with anti-CD3 and their proliferation was similarly conduced as FIG. 4A. (C) EAE was induced by immunizing mice with MOG₃₅₋₅₅ peptide following pertussis toxin administration as detailed in the materials and methods. 2×10⁶ lymphoblast or non-lymphoblast cells were i.v administered to C57/BL6 mice at the time of disease induction. Clinical scores were monitored every other day. (D) On day 26 some of these mice were sacrificed and draining lymph node cells were stimulated with PMA and Ionomycin for 5 hours and BFA for 4 hours for intracellular cytokine staining on CD4⁺ cell gate.

### DETAILED DESCRIPTION OF THE INVENTION

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “a polymerase” refers to one agent or mixtures of such agents, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Although the present invention is described primarily with reference to specific embodiments, it is also envisioned that other embodiments will become apparent to those skilled in the art upon reading the present disclosure, and it is intended that such embodiments be contained within the present inventive methods.

### I. Overview

Foxp3⁺CD4⁺ regulatory T (Treg) cells play a crucial role in maintaining immune homeostasis against self-tissues (Sakaguchi, 2004; Shevach, 2002). These cells also prevent autoimmune and inflammatory diseases through suppressing potentially deleterious activities of T helper (T_{H}) cells. Lack or dysfunction of Treg cells is responsible for many autoimmune diseases (Miyara, 2005; Valencia, 2006; Pop, 2005).

Foxp3, a fork head transcription factor, is expressed mainly in Treg cells and essential for the development and function of Tregs (Khattri, 2003). As Foxp3 is a transcript factor that is expressed in the nucleus, it is not possible to directly isolate viable Foxp3⁺ cells using anti-Foxp3 antibody staining and cell sorting.

By using conventional methods, the highest enrichment for Foxp3⁺ cells achieved in the prior art is about 75%; that is, only 75% of the isolated cell population express Foxp3. Thus, other approaches which are able to specifically identify and enrich for Foxp3 positive cells would be invaluable to study of Tregs and their manipulation and use as therapeutics.

Disclosed herein are methods whereby viable FOXP3⁺ cells can be isolated or enriched on the basis of forward scatter (FSC) and/or side scatter (SSC) properties. Flow cytometric analysis is performed by applying laser beam(s) with a single wavelength to a fluid containing cells and then capturing the light transmitted from the fluid using a plurality of detectors. The FSC signal is the signal detected in line with, or at low angles away from, the direction of the laser beam, while the SSC signal is detected at greater angles away from the direction of the laser beam.

### Flow Cytometry

Flow cytometry is the measurement of cellular properties in solution within a flow system, which delivers the cells singly past a point of measurement.

In general, flow cytometric analysis is performed by applying laser beam(s) with a particular wavelength(s) to a fluid containing cells and then capturing the light transmitted from the fluid using a plurality of detectors.

Lenses collect the laser light coming from cells as a result of their illumination by the laser beam(s). Typically there is one lens in the forward direction, along the path of the laser beam, and one lens at a right angle (orthogonal). In front of the forward lens is a bar, an “obscuration bar,” approximately 1 mm in width, which is positioned so as to block direct laser light. Because of the position of the obscuration bar, only laser light that passes through a cell and is diverted (refracted or scattered) enough from its original direction is collected by the forward lens. Light striking this forward scatter lens is therefore light that has been bent to small angles by the cell. The three dimensional range of angles collected by this lens falls between those obscured by the obscuration bar and those lost at the limits of the outer diameter of the lens. Typically, and without limitation, the range of angles of light collected by the forward scatter lens is between 1° and 10°, that is approximately 1°, 2°, 3°, 4°, 5°, 6°, 7°, 8°, 9°, or 10° and more typically, 1° to 5°. The light striking this lens is referred to as forward scatter (FSC) or forward angle light scatter (FALS).

Although defined in terms of the optics of light collection for any given cytometer, FSC light is not well defined in terms of biology of the cell that generates this light. A cell with a large cross-sectional area will refract a large amount of light. Also, a large cell with a refractive index close to that of the medium, for example, a dead cell, will refract less light than a similarly large cell with a refractive index quite different from that of the medium. Because of this, FSC is often used as a proxy for cell size or volume.

FSC measurements have been used for estimating cell size since it was demonstrated by Mulloney et al. that the intensity of light scattered at small angles from the incident laser beam is roughly proportional to particle volume. However, as described above, a number of cellular properties may influence FSC aside from cell size including differences in refractive index between the cells and the suspending medium, the cell's internal structure, and the presence within or upon cells material with strong absorption at the illumination wavelength used.

In addition to cell size, FSC characteristics have been used to discriminate healthy from diseased cells. Cells with damaged membranes, that is, those cells identifiable as dead by the uptake of dyes such as trypan blue or ethidium bromide, have a lower refractive index and thus produce smaller FSC signals.

The lens at right angles to the direction of the laser beam collects light that has been scattered to wide angles from the original direction. Light collected by this lens is defined by the diameter of the lens and its angle from the point where the laser beam(s) intersects the cells. This light is referred to as side scatter (SSC) light or 90° light scatter. However, while stated in terms of 90° light scatter, as is appreciated by one of skill in the art, it is not required to detect SSC at right angles to the laser beam intersection point, detection of SSC at angles less than 90° can be accomplished. For example, and without limitation, the range of angles of light collected by the side scatter lens is between 10° and 90°, that is approximately 10°, 20°, 30°, 40°, 50°, 60°, 70°, 80°, or 90°.

Light scatter to these angles is often associated with irregularities or texture in the cell surface or cytoplasm of the cell. For example, granulocytes with irregular nuclei scatter more light to the side than do lymphocytes with spherical nuclei. Similarly, more scatter light is produced by fibroblasts than by monocytes.

A number of factors may influence the FSC profile of a cell or a population of cell; these include differences in refractive index between the cells and the suspending medium, the cell's internal structure, and the presence within or upon cells material with strong absorption at the illumination wavelength used. However, one of skill is aware of and can compensate for these limitations when using FSC and SSC properties in identifying a particular cell or cell population.

A method whereby viable Foxp3⁺ cells can be isolated or enriched based on forward scatter (FSC) and/or side scatter (SSC) properties is herein disclosed. Generally each cell type has a unique combination of measured properties, including FSC and SSC signals, which allow the cell type of each cell to be identified. In some embodiments, a population of cells enriched for Foxp3 expression, the “lymphoblast population,” is those cells with high FSC and low SSC relative to the “non-lymphoblast cell population.” The lymphoblast and the non-lymphoblast populations become evident upon cell culture, particularly the culturing of lymphocytes under conditions associated with the expansion of nTregs or the formation of iTregs.

An “enriched Foxp3′ sample” or “lymphoblast population” refers to those samples that have been enriched for Foxp3′ cells by selection of cells based on FSC and/or SSC, or any combination of these properties with cell surface markers, for example CD4 and/or CD25. An enriched sample is one in which more than 75%, 80%, 85%, 90%, 95%, 99% or more cells express Foxp3. A “non-lymphoblast population” refers to those samples based on FSC and/or SSC, or any combination of these properties with cell surface markers, for example CD4 and/or CD25 wherein less than 80% of the cells express Foxp3.

Methods for detecting Foxp3 expression in isolated cell populations are known to those of skill in the art, and include, without limitation, intracellular staining with anti-Foxp3 reagents, for example, antibodies and nucleic acid probes.

Many flow cytometers are also “cell sorters”, which have the ability to selectively deposit cells from particular populations into tubes, or other collection vessels. This procedure is well known in the art and described by, for example, Melamed, et al. Flow Cytometry and Sorting Wiley-Liss, Inc., New York, N.Y. (1990); Shapiro Practical Flow Cytometry, 4 ed, Wiley-Liss, Hoboken, N.J. (2003); and Robinson et al. Handbook of Flow Cytometry Methods Wiley-Liss, New York, N.Y. (1993); Harkins and Galbraith (1987) and U.S. Pat. No. 4,765,737.

In order to sort cells, the instruments electronics interprets the signals collected for each cell as it is interrogated by the laser beam and compares the signal with sorting criteria set on the computer, the gate. If the cell meets the required criteria, an electrical charge is applied to the liquid stream which is being accurately broken into droplets containing the cells. This charge is applied to the stream at the precise moment the cell of interest is about to break off from the stream, then removed when the charged droplet has broken from the stream. As the droplets fall, they pass between two metal plates, which are strongly positively or negatively charged. Charged droplets get drawn towards the metal plate of the opposite polarity, and deposited in the collection vessel, or onto a microscope slide, for further examination.

The cells can automatically be deposited in collection vessels as single cells or as a plurality of cells, e.g. using a laser, e.g. an argon laser (488 nm) and for example with a flow cytometer fitted with an Autoclone unit (Coulter EPICS Altra, Beckman-Coulter, Miami, Fla., USA). Other examples of suitable FACS machines useful for the methods of the invention include, but are not limited to, MoFlo™. High-speed cell sorter (Dako-Cytomation Ltd), FACS Aria™ (Becton Dickinson), ALTRA™. Hyper sort (Beckman Coulter) and CyFlow™ sorting system (Partec GmbH).

While one of skill in the art could identify the lymphoblast population based on FSC and SSC properties, appropriate controls can be used to aid in identifying the lymphoblast population.

For example, mice in which the nucleic acid encoding green fluorescent protein (GFP) is inserted into the Foxp3 gene are known, Foxp3^{gfp} knock-in mice. Such mice express GFP when Foxp3 is expressed. Cells from these mice can be cultured and applied to a flow cytometer. The population of cells with 80% or more of the cells expressing GFP with the relatively highest FSC and relatively lowest SSC identify the Foxp3+ lymphoblast population. Once the relative position of the lymphoblast population is identified using Foxp3^{gfp} knock-in mice, samples from non-Foxp3^{gfp} knock-in animals can be applied to the flow cytometry subsequently and identified.

Other controls known by those of skill in the art include, without limitation, beads of defined and differing sizes for flow cytometric analysis. Such methods are described for example in U.S. Pat. No. 5,084,394, incorporated by reference in its entirety herein.

For example, defined size beads can be added with an expanded nTreg population. Beads of one particular size or range of sizes can be identified as co-segregating with the lymphoblast population, for example on the basis of co-segregating with lymphoblast cells from Foxp3^{gfp} knock-in mice or populations identified as possessing enhanced suppressive activity described below. In subsequent experiments, the lymphoblast population would be identified as those cells co-segregating with beads of the appropriate size.

Identification of the Lymphoblast Population from Expanded nTregs

In some embodiments, the lymphoblast population is isolated from nTregs expanded in vitro. nTregs are thymus derived regulatory T cells and can be isolated from lymphoid tissues, for example, the thymus, spleen, lymph nodes or from bodily fluids, for example, blood. nTregs can be obtained from any mammalian subject, including mice and humans.

Methods for expanding nTregs are known in the art. Such methods often entail, without limitation, the culturing of a starting population of isolated cells under appropriate culture conditions. Often the isolated cells are cells that are CD4⁺ and/or CD25⁺.

In some embodiments, the lymphoblast population and/or the non-lymphoblast populations develop and can be isolated by expanding isolated CD4⁺CD25⁺ in the presence of anti-CD3, anti-CD28, and/or IL-2. These cells can be maintained in vitro over the course of 1, 2, 3, 4, 5, 6, 7, or more days. Cells resulting from this process are referred to as “expanded nTregs.”

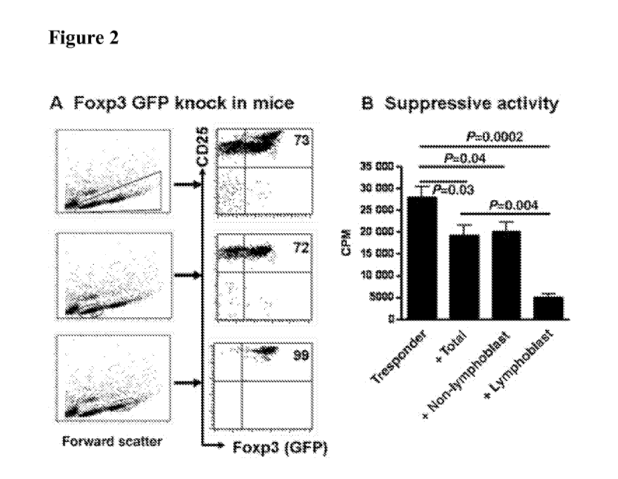

Analysis of these cultured cells by flow cytometry reveals a distinctive dot plot profile as shown in FIG. 2A. Surprisingly, the highest percentage of cells expressing Foxp3 are those cells with relatively high FSC and relatively low SSC.

The lymphoblast population is the sub-population of cells that are the 80%, 75%, 70%, 65%, 60%, 55%, 50%, 45%, 40%, 35%, 30%, 20%, 15%, 10% of cells identified as having the highest FSC among the expanded nTregs and/or the 50%, 45%, 40%, 35%, 30%, 25%, 20%, 15%, 10%, 5%, or less of cells with the lowest SSC.

Accordingly, the FSC and SSC properties of a cell can be used to identify and enrich for those cells which express FOXP3⁺.

In addition to expanded nTregs, the lymphoblast population and/or non-lymphoblast population can be isolated from iTregs.

Identification of the Lymphoblast Population from Expanded iTregs

In some embodiments, the lymphoblast population is isolated from iTregs. iTregs are naïve T cells and can be isolated from lymphoid tissues, for example, the thymus, spleen, lymph nodes or from bodily fluids, for example, blood. iTregs can be obtained from any mammalian subject, including mice and humans.

Methods for making iTregs are known in the art. Such methods often entail, without limitation, the culturing of a starting population of isolated cells under appropriate culture conditions. Often the isolated cells are CD4⁺.

In some embodiments, the lymphoblast population and/or the non-lymphoblast populations develop and can be isolated by culturing isolated CD4⁺ cells in the presence of anti-CD3, anti-CD28, IL-2, and/or TGF-β. Cells resulting from this process are referred to as “iTregs.”

The phenotype of the lymphoblast population of iTregs is similar, if not the same, as that of nTregs.

### Diseases and Disorders

Populations of enriched Foxp3⁺ cells, by methods of such as FACS, can be used in the treatment of diseases and disorders.

In certain aspects, the present invention provides methods and compositions for the prevention and treatment of immune conditions; that is, those diseases, disorders and reactions or responses wherein the immune system contributes to pathogenesis.

As used herein, the term “sample” or “biological sample” refers to tissues or bodily fluids removed from a mammal, preferably human, and which contain regulatory T cells. In some embodiments, the samples are taken from individuals with an immune response which needs to be suppressed. In some embodiments, the individual has an allergy, Graft vs. Host Disease, an organ transplant, or autoimmune disorder. Samples preferably are blood and blood fractions, including peripheral blood. The biological sample is drawn from the body of a mammal, such as a human, and may be blood, bone marrow cells, or similar tissues or cells from an organ afflicted with the unwanted immune response. Methods for obtaining such samples are well known to workers in the fields of cellular immunology and surgery. They include sampling blood in well known ways, or obtaining biopsies from the bone marrow or other tissue or organ. In preferred embodiments, the sample is a T-cell enriched sample in which the sample cells are substantially T-cells.

Immune conditions also include autoimmunity. Autoimmunity is the persistent and progressive immune reactions to non infectious self antigens, as distinct from infectious non self antigens from bacterial, viral, fungal, or parasitic organisms which invade and persist within mammals and humans.

An “autoimmune disease” refers to a disease associated with the inability of the immune system to discriminate between self and non-self. Examples of autoimmune diseases include, without limitation, immune dysregulation, polyendocrinopathy, enteropathy, X-linked (IPEX), type 1 diabetes, arthritis (including rheumatoid arthritis, juvenile rheumatoid arthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia greata, allergic responses due to arthropod bite reactions, Crohn's disease, iritis, conjunctivitis, keratoconjunctivitis, ulcerative colitis, asthma, allergic asthma, cutaneous lupus erythematosus, scleroderma, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

A patient with an autoimmune disease may be diagnosed as known to one of ordinary skill in the art. Such patients may be identified symptomatically and/or by obtaining a sample from a patient and isolating autoreactive T cells and comparing the level of autoreactive T cells in a patient to a control (see, U.S. Patent Application Publication No. 20060105336). For instance, type 1 diabetes may be identified by age of on-set and dependence on insulin injections to maintain glucose homeostasis.

The response of a patient with an autoimmune disease to treatment may be monitored by determining the severity of their symptoms or by determining the frequency of autoreactive T cells in a sample from a patient with an autoimmune disease. The severity of symptoms of the autoimmune disease may correlate with the number of autoreactive T cells (see, U.S. Patent Application Publication No. 20060105336). In addition, an increase in the number of autoreactive T cells in the sample may be used as an indication to apply treatments intended to minimize the severity of the symptoms and/or treat the disease before the symptoms appear.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### EXAMPLES

### Example 1

### FACS Scatter Characteristics Define a Population Enriched for Foxp3⁺ Cells

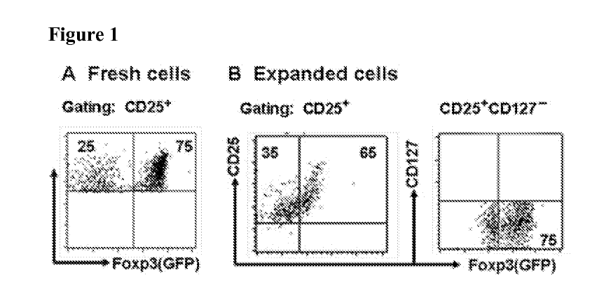

Conventional methods for enriching for Treg cells employ a combination of CD4 and CD25 surface marker staining. In order to analyze how effective this scheme is in enriching for Foxp3⁺ cells, Foxp3^{gfp} knock-in mice were used. Foxp3^{gfp} knock-in mice possess a green fluorescence protein (GFP) inserted within the Foxp3 gene. Using this combination of markers, CD4, CD25, and GFP, revealed that only about 75% of CD4⁺CD25⁺ were positive for GFP (FIG. 1A). Gating on CD127-(hIL-7R-M21) cells did not markedly improve this percentage. Thus, many of the cells isolated by the combination of CD4 and CD25 presumably do not express Foxp3.

In many instances, CD4⁺CD25⁺ cells are isolated and expanded in vitro. It was of interest to determine whether the expansion of such cells in culture would result in an increase in the proportion of cells positive for GFP, and in turn Foxp3. In order to accomplish this, splenic T cells from Foxp3^{gfp} knock-in mice were sorted on the basis of CD4⁺CD25⁺ expression using a FACSAria (BD). FACS isolated CD4⁺CD25⁺ spleenocytes were placed in culture with anti-CD3 and anti-CD28-coated beads (1:5, one bead to five cells) along with IL-2 (rmIL-2; 200 U/ml; R&D) for 4 days. After this period, the expression of Foxp3′ and the presence of other markers were assessed. FACS analysis of these cells revealed that while CD25 expression was maintained, expression of CD127 was almost completely lost. Foxp3 expression on the CD25′ cell population was slightly decreased following cell expansion in vitro. Gating on CD25⁺CD127⁻ cell population did not significantly improve the percentage of Treg cells since 25% of these cells were still GFP negative (FIG. 1B).

It was noticed that after four days in culture, in the presence of anti-CD3 and anti-CD28-coated beads along with IL-2, two distinct populations were clearly distinguishable on the basis of FSC and SSC characteristics. One population, the “lymphoblast population,” representing about 45% of total viable cells, and another one referred to as a “non-lymphoblast cell population.” Gates drawn to encompass these populations are shown in FIG. 2. The uppermost scatter dot plot depicts a gate drawn to encompass viable lymphoblast and non-lymphoblast cell populations. The middle scatter dot plot is drawn to encompass the non-lymphoblast cell population, while the lower most scatter dot plot is drawn to encompass the lymphoblast cell population.

While the total population of expanded cells expressed similar levels of Foxp3 with that of freshly isolated CD4⁺CD25⁺ cells (about 70%), more than 98% of the lymphoblast cell population expressed Foxp3 and their CD25 and Foxp3 mean fluorescent intensity were significantly higher than either the total cell population or non-lymphoblast cell populations. The non-lymphoblast cell population was composed of approximately 70% Foxp3⁺ and approximately 30% Foxp3⁻ cells.

Of interest was whether the lymphoblast and non-lymphoblast populations could be identified in mice in which the Foxp3 gene had not been manipulated. To accomplish this, CD4⁺ CD25⁺ cells were isolated and cultured in the presence of anti-CD3/CD28 coated beads and IL-2 for four days as described above. As with the Foxp3^{gfp} knock-in mice, lymphoblast and non-lymphoblast cell populations could be resolved on the basis of FACS scatter properties. Similar levels of Foxp3 expression, between the lymphoblast and non-lymphoblast cell populations, to that of the Foxp3^{gfp} knock-in mice were also observed. Thus, the enrichment for Foxp3 bearing cells based on scatter characteristics is not limited to Foxp3^{gfp} knock-in mice.

### Example 2

### Isolated Lymphoblast Population Possesses Suppressive Activity

To measure the in vitro suppressive assay, T cells isolated from Foxp3^{gfp} knock-in mice were stimulated with soluble anti-CD3 (0.25 μg/ml) in the presence of γ-irradiated APC (1:1) with or without lymphoblast or non-lymphoblast cell population in the 1:4 ratio (1 Treg subset to 4 T responder). ³H-thymidine (1 μCi/96 well) was added to cultures in the last 16 hours and cell proliferation was measured by using a liquid scintillation counter. As shown in FIG. 2B, while expanded nTreg cells harvested from total or sorted from non-lymphoblast cell populations significantly suppressed T cell proliferation, expanded nTreg cell sorted from the lymphoblast population displayed more potent suppressive activity. In fact, Foxp3⁻ (GFP⁻) cells sorted from expanded CD4⁺CD25⁺ cells exhibited little suppressive ability.

### Example 3

### The Lymphoblast Population is also found in Induced Tregs

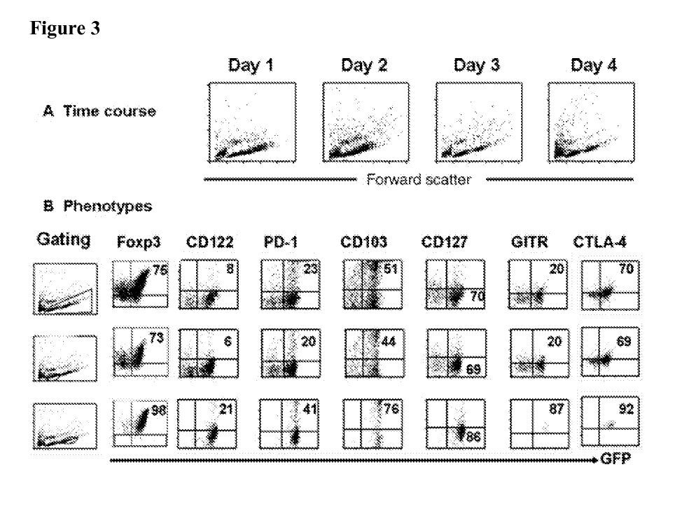

The combination of IL-2 and TGF-β is able to induce CD4⁺Foxp3⁺ iTregs that are similar in phenotype and functional characteristics with nTregs. Whether the lymphoblast population could be identified in iTreg was explored. Naïve CD4⁺GFP⁻ cells stimulated with anti-CD3/CD28 coated beads and IL-2 and TGF-β. As is shown in FIG. 3, after three days under these culture conditions, two cell populations, akin to the lymphoblast and non-lymphoblast population were witnessed. Compared with total lymphocyte and non-lymphoblast populations, the lymphoblastic iTregs expressed significantly higher levels of Foxp3, CD25, CD103, CD122, PD1, GITR, and CTLA-4 and lower levels of CD127 (FIG. 3B), similar to the characteristics of purified Treg cells.

### Example 4

### The Lymphoblast Cell Population Display Potent Suppressive Activity in an EAE Model

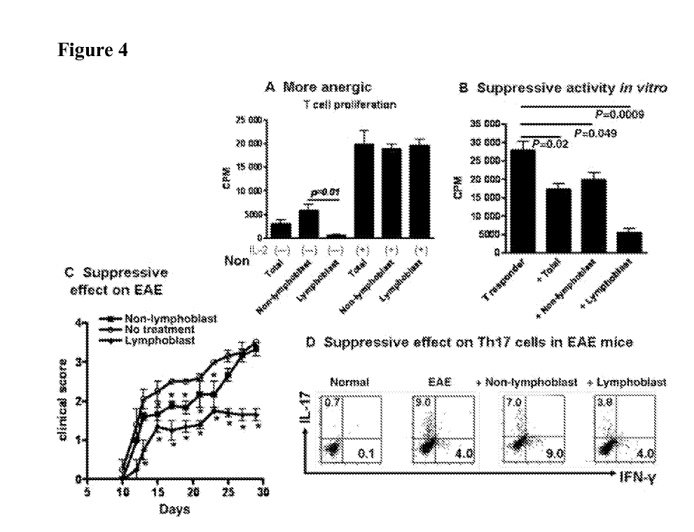

EAE was induced in C57B1/6 mice by immunization with MOG₃₅₋₅₅ emulsified in CFA (Difco Laboratories) at a dose of 100 μg per mouse, followed by the administration of pertussis toxin (150 ng per mouse; Sigma) on days 0 and 2 as described (Stromnes and Goverman, 2006). 2×10⁶ lymphoblast cell and non-lymphoblast cell population sorted form iTregs were adoptively transferred to mice at the time of disease induction. Clinical signs of EAE were assigned scores according to the following: 0, no symptoms; 1, loss of muscle tone in tail; 2, hind limp weakness; 3, hind limp paralysis of one (3.0) or both (3.5); 4, hind and fore limp paralysis; 5, loss of temperature control or moribund. Scores are shown as Mean daily clinical scores for all mice per group (FIG. 4).

Mice were sacrificed 26 days after MOG₃₅₋₅₅ peptide immunization with or without Treg subset injection. Draining lymph nodes (LN) were collected and single cell suspension were prepared from LN and stimulated with PMA (50 ng/ml) and ionomycin (100 ng/ml) for 5 h and brefeldin A (5 μg/ml) for 4 h. Cells were stained for surface CD4, fixed, permeabilized, and then stained for intracellular IL-17 and IFN-γ. These results show that adoptive transfer of the lymphoblast cell population results in persistent suppression of autoimmune disease.

The present specification provides a complete description of the methodologies, systems and/or structures and uses thereof in example aspects of the presently-described technology. Although various aspects of this technology have been described above with a certain degree of particularity, or with reference to one or more individual aspects, those skilled in the art could make numerous alterations to the disclosed aspects without departing from the spirit or scope of the technology hereof. Since many aspects can be made without departing from the spirit and scope of the presently described technology, the appropriate scope resides in the claims hereinafter appended. Other aspects are therefore contemplated. Furthermore, it should be understood that any operations may be performed in any order, unless explicitly claimed otherwise or a specific order is inherently necessitated by the claim language. It is intended that all matter contained in the above description and shown in the accompanying drawings shall be interpreted as illustrative only of particular aspects and are not limiting to the embodiments shown. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all published references and patent documents referred to in this disclosure are incorporated herein by reference in their entirety for all purposes. Changes in detail or structure may be made without departing from the basic elements of the present technology as defined in the following claims.

We claim:

1. A method for enriching for Foxp3 expressing cells, said method comprising providing a population of lymphocytes, isolating from among said population of lymphocytes those cells with relatively high forward scatter and relatively low side scatter, and thereby enriching for Foxp3 expressing cells.

2. The method of claim 1, wherein said population of lymphocytes is cultured in a medium prior to isolating those cells with relatively high forward scatter and relatively low side scatter.

3. The method of claim 2, wherein said medium comprises IL-2.

4. The method of claim 2, wherein said medium comprises TGF-β.

5. The method of claim 2, wherein said medium comprises anti-CD3 and anti-CD28 beads and IL-2.

6. The method of claim 1, wherein said population of lymphocytes is sorted on the basis of CD4 and CD25 expression prior to isolating those cells with relatively high forward scatter and relatively low side scatter.

7. The method of claim 6, wherein 75% of the resulting cells express Foxp3.

8. The method of claim 6, wherein 85% of the resulting cells express Foxp3.

9. The method of claim 6, wherein 95% of the resulting cells express Foxp3.

10. A method for treating a subject in need thereof, said method comprising providing a population of lymphocytes, isolating from among said population of lymphocytes those cells with relatively high forward scatter and relatively low side scatter, thereby enriching for a population of Foxp3 expressing cells, administering said population of Foxp3 expressing cells to said subject in need thereof.

11. A method for enriching for Foxp3 expressing cells, said method comprising providing a population of lymphocytes, isolating from said population of lymphocytes those cells expressing CD25 and CD4, thereby forming a precursor population, culturing said precursor population in a medium, isolating from among said precursor population those cells with relatively high forward scatter and relatively low side scatter, and thereby enriching for Foxp3 expressing cells.

12. The method of claim 11, wherein said medium comprises anti-CD3 and anti-CD28 beads and IL-2.

13. The method of claim 11, wherein said medium comprises anti-CD3 and anti-CD28 beads, IL-2, and TGF-β.

14. The method of claim 11, wherein the precursor population is cultured for at least three days.

15. A composition of cells, wherein 80% or more of the cells express Foxp3.

16. The composition of claim 15, wherein 85% or more of the cells express Foxp3.

17. The composition of claim 15, wherein 90% or more of the cells express Foxp3.

18. The composition of claim 15, wherein 95% or more of the cells express Foxp3.
